(19) United States
(12) Patent Application Publication
(10) Pub. No.: US 2025/0255933 A1
(43) Pub. Date: Aug. 14, 2025

(54) Modification of Immune Cells to Increase Activity

(71) Applicant: The Regents of the University of California, Oakland, CA (US)

(72) Inventors: Dan Kaufman, La Jolla, CA (US); Huang Zhu, La Jolla, CA (US)

(21) Appl. No.: 19/010,630

(22) Filed: Jan. 6, 2025

Related U.S. Application Data
(63) Continuation of application No. 18/649,526, filed on Apr. 29, 2024, now abandoned.
Application No. 18/649,526 is a continuation of application No. 17/047,515, filed on Oct. 14, 2020, now abandoned.
(60) Provisional application No. 62/670,033, filed on May 11, 2018.

(51) Int. Cl. A61K 38/20; A61K 40/15; A61K 40/42; C12N 5/0783; C12N 15/113

(52) U.S. Cl. CPC A61K 38/2013; A61K 38/2086; A61K 40/15; A61K 40/42; C12N 5/0646; C12N 15/113; A61K 2239/38; A61K 2239/48; C12N 2310/20; C12N 2501/42; C12N 2510/00

(57) ABSTRACT

Compositions, methods of making, and using modified immune cells such as NK cells to treat cancer, viral and microbial infection. The modified CISH^{-/-} NK cells exhibit hypersensitivity to cytokines such as IL-2 and/or IL-15 and maintain expansion and anti-tumor functions.

### CROSS REFERENCE TO RELATED APPLICATIONS

This application is a continuation of Ser. No. 18/649,526, filed Apr. 29, 2024, which is a continuation of Ser. No. 17/047,515, filed Oct. 14, 2020, which claims the priority benefit of PCT/US2019/031979, filed on May 13, 2019, which claims the priority benefit to U.S. Provisional Patent Application No. 62/670,033, filed May 11, 2018, the entire contents of which are incorporated herein by reference.

### STATEMENT OF GOVERNMENT INTEREST

This invention was made with support from the National Institutes of Health under Grant Nos. CA217885 and CA203348. The government has certain rights in the invention.

### BACKGROUND

Natural killer (NK) cells are a critical part of the innate immune system, and are an important effector of lymphocyte population in anti-tumor and anti-infection immunity. However, tumor progression and chronic infections generally causes NK cell exhaustion, resulting in poor effector function and limiting the anti-tumor/infection potential of NK cells. The exact mechanisms leading to NK cell exhaustion in tumors and chronic infections are poorly defined.

The detection of aberrant cells by NK cells is controlled by activating and inhibitory signals from ligands and cytokines such as interleukin-15 (IL-15). Cytokine-inducible SH2-containing protein (CIS) is a critical negative regulator of IL-15 signaling in NK cells which is encoded by CISH gene in human. CISH is rapidly induced in response to IL-15, and the deletion of the CISH gene has been shown to increase the sensitivity of NK cells to IL-15. Recent studies in mice have demonstrated that CIS is a potent inhibitory checkpoint in NK cell-mediated tumor immunity.

NK cells need cytokines, such as interleukin-2 (IL-2) and IL-15, to maintain activity and function, however IL-2 causes systemic toxicity. Thus, there remains a need for clinical NK cell therapy for treatment of cancers, and other diseases, that maintains expansion and function without cytokines or only requires low cytokine doses.

### SUMMARY OF THE INVENTION

The disclosure generally provides compositions and methods for using CISH^{-/-} modified NK cells in cancer treatment. The modified NK cells exhibit hypersensitivity to IL-2 and/or IL-15 stimulation and can maintain expansion and anti-tumor functions with low concentration cytokines or growth factors, such as interleukins.

According to one aspect of the present disclosure, there is provided a CISH^{-/-} modified NK cell usable as a cell source of NK cell-based therapy for treatment of cancers and other diseases or infections with improved therapeutic effects over unmodified native NK cells.

According to one aspect of the present disclosure, there is provided a method for the manufacture of CISH^{-/-} NK cells.

According to another aspect of the present disclosure, there is provided a cell culture of CISH^{-/-} NK cells, and pharmaceutical compositions comprising CISH^{-/-} NK cells.

### BRIEF DESCRIPTION OF THE DRAWINGS

A better understanding of the features and advantages of the present disclosure will be obtained by reference to the following detailed description that sets forth illustrative embodiments, in which the principles of the disclosure are utilized, and the accompanying drawings of which:

FIGS. 1A-1C depict the effect of loss of CISH on NK differentiation using a regular method.

FIGS. 2A-2B depict the effect of loss of CISH on NK differentiation using a modified method.

FIGS. 3A-3B depict CISH^{-/-} NK cell expansion.

FIGS. 4A-4B depict the result from an incucyte killing assay.

FIGS. 5A-5C depict CISH^{-/-} iPSC-NK cells show higher single cell polyfunctional response upon cytokine stimulation.

FIGS. 6A-6C depict CISH^{-/-} iPSC-NK cells show increased basal glycolysis and glycolytic capacity.

FIGS. 7A-7C depict CISH^{-/-} iPSC-NK cells mediate better anti-tumor activity in human leukemia systemic tumor model.

### DETAILED DESCRIPTION

All publications, patents, and patent applications mentioned in this specification are herein incorporated by reference to the same extent as if each individual publication, patent, or patent application was specifically and individually indicated to be incorporated by reference. Citations to publications are intended to reference the most current edition thereof.

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as Molecular Cloning: A Laboratory Manual, second edition (Sambrook et al., 1989) Cold Spring Harbor Press; Oligonucleotide Synthesis (M J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and C C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (CA. Janeway and P. Travers, 1997); Antibodies (P. Finch, 1997); Antibodies: a practical approach (D. Catty., ed., IRL Press, 1988-1989); Monoclonal antibodies: apractical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J.B. Lippincott Company, 1993).

The present invention relates to a method for treating a diseases, such as cancer, or an infection caused by, for example, a virus or bacteria, in a human subject, comprising administering to a human subject in need an effective amount of a pharmaceutical composition comprising human CISH^{-/-} natural killer (NK) cells and a pharmaceutically acceptable carrier.

In embodiments, the present invention relates to a method for treating a cancer in a human subject, wherein said NK cells are derived from human induced pluripotent stem cells (iPSCs), embryonic stem cells (ESCs), or peripheral blood cells.

In embodiments, the present invention relates to a method for treating a cancer or infection in a human subject, wherein the CISH^{-/-} NK cells are autologous to the subject.

In embodiments, the present invention relates to a method for treating a cancer in a human subject, wherein the method further comprises administering to the subject an effective amount of a cytokine, such as IL-2, IL-15 or both.

In embodiments, the present invention relates to a method for treating a cancer in a human subject, wherein the effective amount of IL-2 and/or IL-15 is less than an effective amount required with native NK cell treatment. In embodiments, the low concentration of IL-2 is between 1 and 10 U/ml, or about 5 U/ml, and the low concentration of IL-15 is between 1 and 10 ng/ml, or about 5 ng/ml, which is effective to maintain CISH^{-/-} NK cell expansion and anti-tumor functions.

Cytokines that can be used in the present invention include naturally occurring, modified and synthetically engineered cytokines and cytokine-like molecules (such as ALT-803 or NEKTAR Therapeutics, Inc. products such as NKTR-358 or NKTR-255). Cytokines include interleukins such as IL-2, IL-12, IL-15, IL-18, IL-21.

In embodiments, the present invention relates to a method for treating a cancer in a human subject, wherein the cancer is hematopoietic or a solid tumor.

In embodiments, the present invention relates to a method for treating a disease or infection in a human subject, wherein the CISH^{-/-} NK cells are hypersensitive to cytokine stimulation and demonstrate improved expansion, anti-tumor function, and anti-viral function as compared to native NK cells.

In embodiments, the present invention relates a pharmaceutical composition comprising human CISH^{-/-} NK cells, and at least one pharmaceutically acceptable excipient.

In embodiments, the present invention relates a pharmaceutical composition, wherein the CISH^{-/-} NK cells are hypersensitive to cytokine stimulation and demonstrate improved expansion, anti-tumor function, and anti-viral function as compared to native NK cells.

In embodiments, the present invention relates a pharmaceutical composition, wherein the cytokine stimulation comprises stimulation with an interleukin, such as IL-2 and/or IL-15. In embodiments, the low concentration of IL-2 is between 1 and 10 U/ml, or about 5 U/ml, and the low concentration of IL-15 is between 1 and 10 ng/ml, or about 5 ng/ml, which is effective to maintain CISH^{-/-} NK cell expansion and anti-tumor functions.

In embodiments, the present invention relates a pharmaceutical composition, wherein the CISH^{-/-} NK cells are derived from induced pluripotent stem cells, embryonic stem cells, or peripheral blood cells.

In embodiments, the present invention relates to a method for producing CISH^{-/-} NK cells comprising: deleting the CISH gene from human induced pluripotent stem cells (iPSCs) or embryonic stem cells (ESCs); and deriving NK cells from the CISH^{-/-} iPSCs using an in vitro differentiation protocol.

In embodiments, the present invention relates to a method for producing CISH^{-/-} NK cells, wherein the deletion of the CISH gene is achieved by using a CRISPR system such as the CRISPR/Cas9 system.

In embodiments, the present invention relates to a method for producing CISH^{-/-} NK cells, wherein the deriving step further comprises differentiating the CISH^{-/-} iPSCs to >75%, >60%, >70%, or >80% CD34⁺, and then differentiating to >75%, >60%, >70%, or >80% CD45⁺ and CD56⁺.

In embodiments, the present invention relates to a method for producing CISH^{-/-} NK cells, wherein the second differentiation occurs in contact with Notch ligand, for example with OP9-DL4 cells which are engineered to over-express Notch ligand.

In embodiments, the present invention relates to a method for producing CISH^{-/-} NK cells, wherein a cell culture comprises CISH^{-/-} NK cells.

### Definitions

To facilitate understanding of the invention, a number of terms and abbreviations as used herein are defined below as follows:

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

It is understood that aspects and embodiments of the invention described herein include “consisting” and/or “consisting essentially of” aspects and embodiments.

It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Values or ranges may be also be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In embodiments, “about” can be used to mean, for example, within 10% of the recited value, within 5% of the recited value, or within 2% of the recited value.

As used herein, “patient” or “subject” means a human or animal subject to be treated.

As used herein, “proliferation” or “expansion” refers to the ability of a cell or population of cells to increase in number.

As used herein, a composition containing a “purified cell population” or “purified cell composition” means that at least 30%, 50%, 60%, typically at least 70%, and more preferably 80%, 90%, 95%, 98%, 99%, or more of the cells in the composition are of the identified type.

As used herein, “therapeutically effective” refers to an amount of NK cells that is sufficient to treat or ameliorate, or in some manner reduce the symptoms associated with a disease, such as cancer, or condition, such as an infection. When used with reference to a method, the method is sufficiently effective to treat or ameliorate, or in some manner reduce the symptoms associated with a disease or condition. For example, an effective amount in reference to a disease is that amount which is sufficient to block or prevent its onset; or if disease pathology has begun, to palliate, ameliorate, stabilize, reverse or slow progression of the disease, or otherwise reduce pathological consequences of the disease. In any case, an effective amount may be given in single or divided doses.

As used herein, the term “treatment” embraces at least an amelioration of the symptoms associated with a disease or condition in the patient, where amelioration is used in a broad sense to refer to at least a reduction in the magnitude of a parameter, e.g. a symptom associated with the condition being treated. As such, “treatment” also includes situations where the disease, disorder, or pathological condition, or at least symptoms associated therewith, are completely inhibited (e.g. prevented from happening) or stopped (e.g. terminated) such that the patient no longer suffers from the condition, or at least the symptoms that characterize the condition.

As used herein the term “pharmaceutical composition” refers to a pharmaceutical acceptable compositions, wherein the composition comprises NK cells, and in some embodiments further comprises a pharmaceutically acceptable carrier. In some embodiments the pharmaceutical composition may be a combination.

As used herein the term “pharmaceutically acceptable” means approved by a regulatory agency of the Federal or a state government or listed in the U.S. Pharmacopoeia, other generally recognized pharmacopoeia in addition to other formulations that are safe for use in animals, and more particularly in humans and/or non-human mammals.

As used herein the term “pharmaceutically acceptable carrier” refers to an excipient, diluent, preservative, solubilizer, emulsifier, adjuvant, and/or vehicle with which NK cells, are administered. Such carriers may be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose may also be a carrier. Methods for producing compositions in combination with carriers are known to those of skill in the art. In some embodiments, the language “pharmaceutically acceptable carrier” is intended to include any and all solvents, dispersion media, coatings, isotonic and absorption delaying agents, and the like, compatible with pharmaceutical administration. The use of such media and agents for pharmaceutically active substances is well known in the art. See, e.g., Remington, The Science and Practice of Pharmacy, 20th ed., (Lippincott, Williams & Wilkins 2003). Except insofar as any conventional media or agent is incompatible with the active compound, such use in the compositions is contemplated.

The term “combination” refers to either a fixed combination in one dosage unit form, or a kit of parts for the combined administration where NK cells and a combination partner (e.g., another drug as explained below, also referred to as “therapeutic agent” or “co-agent”) may be administered independently at the same time or separately within time intervals. In some circumstances the combination partners show a cooperative, e.g., synergistic effect. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g., a compound and a combination partner, are both administered to a patient simultaneously in the form of a single entity or dosage. The term “non-fixed combination” means that the active ingredients, e.g., a compound and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more active ingredients.

Cytokine Inducible SH2 Containing Protein (CIS) plays a key role in regulating human natural killer (NK) cell activation-induced exhaustion and unlike studies in the murine system, CISH-deletion (CISH^{-/-}) leads to decreased NK cell activity. The presently disclosed model of CISH-deletion in human induced pluripotent stem cells (iPSCs) provides a model to further dissect CISH mediated regulation of human NK cell development, function, activation, persistence, and exhaustion. In other embodiments, deletion of the CISH gene occurs in human embryonic stem cells (hESCs). In embodiments, T cells are derived from CISH^{-/-} iPSC or hESC. Provided herein are compositions and methods for regulating immune cell, such as NK cell or T cell development and for inhibiting immune cell exhaustion.

The invention provides that CISH^{-/-} NK exhaustion can be prevented or inhibited by culturing cells with Notch ligand, such as with a culture layer of OP9-DL4 cells which over-express Notch ligand. Alternative Notch ligand sources are known and include cell-bound or plate bound/cell-free materials.

The present disclosure is based in part on a genome editing tool such as the clustered regularly interspaced short palindromic repeats (CRISPR) system that can be used in a wide variety of organisms (e.g., used to add, disrupt, or change the sequence of specific genes). The CRISPR/Cas9 system is based on two elements. The first element, Cas9, is an endonuclease that has a binding site for the second element, which is the guide polynucleotide (e.g., guide RNA). The guide polynucleotide (e.g., guide RNA) directs the Cas9 protein to double stranded DNA templates based on sequence homology. The Cas9 protein then cleaves that DNA template. By delivering the Cas9 protein and appropriate guide polynucleotides (e.g., guide RNAs) into a cell, the organism's genome is cut at a desired location. Following cleavage of a targeted genomic sequence by a Cas9/gRNA complex, one of two alternative DNA repair mechanisms can restore chromosomal integrity: 1) non-homologous end joining (NHEJ) which generates insertions and/or deletions of a few base-pairs (bp) of DNA at the gRNA cut site, or 2) homology-directed repair (HDR) which can correct the lesion via an additional “bridging” DNA template that spans the gRNA cut site. Further aspects of the CRISPR/Cas system known to those of ordinary skill are described in PCT Publication No. WO 2017/049266, the entire contents of which are hereby incorporated by reference. These and other well-known and new techniques, such as TALEN, for making CISH^{-/-} NK cells are contemplated by the present invention. The invention also contemplates compositions, methods of use and methods of manufacture with hematopoietic cells such as NK cells, T cells and other immune cells.

EXAMPLES

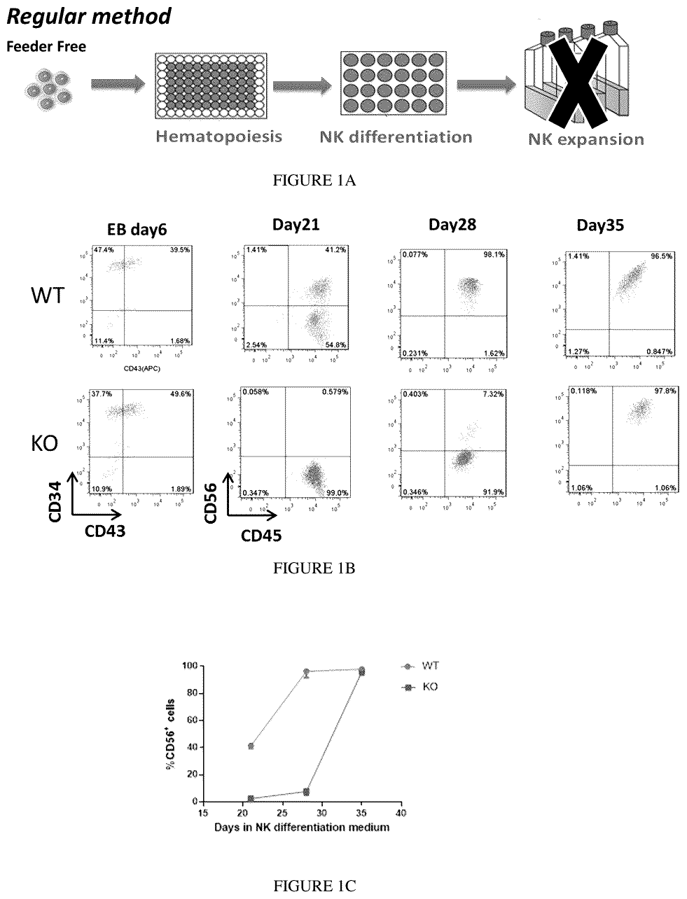

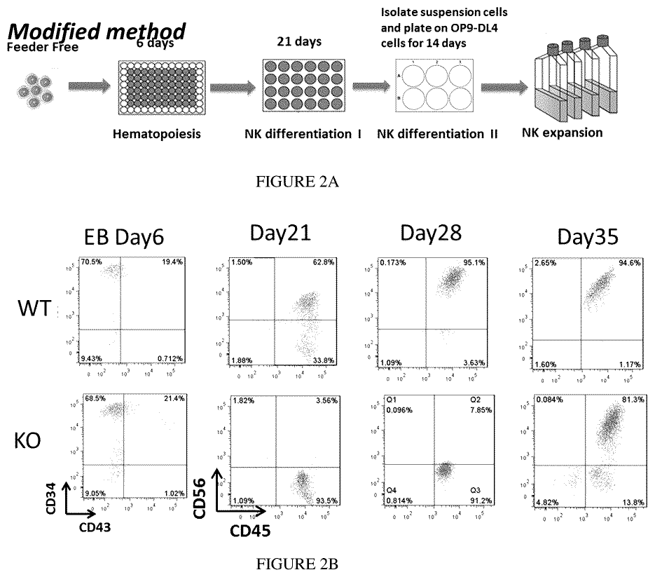

The CISH gene in human induced pluripotent stem cells (iPSCs) was disrupted using the CRISPR/Cas9 system and NK cells from CISH^{-/-} iPSCs were derived using a two-stage in vitro differentiation protocol. The first stage of differentiation into hematopoietic progenitor cells was normal (>80% CD34⁺ cells) using either WT or CISH^{-/-} iPSCs. Deletion of CISH in iPSCs delayed the second stage of in vitro NK cell differentiation (FIGS. 1 and 2). Specifically, whereas NK cell differentiation is typically fully complete with >90% NK cells after 4 weeks using WT iPSCs, the CISH^{-/-} iPSC-derived cells only produced 10% CD45⁺CD56⁺ NK cells at 4 weeks, though by 5 weeks were >80% NK cells. After this time, CISH^{-/-} iPSC-derived NK cells were phenotypically mature and showed typical NK surface maker expression such as CD94, CD16, NKG2D, NKp44, NKp46.

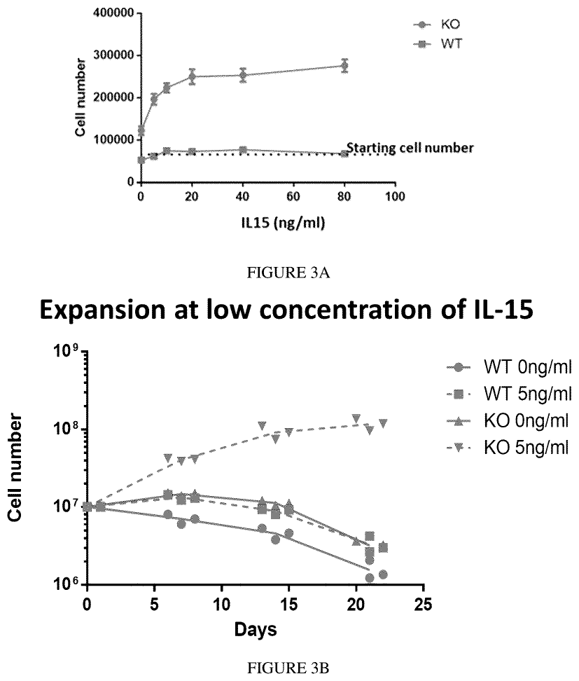

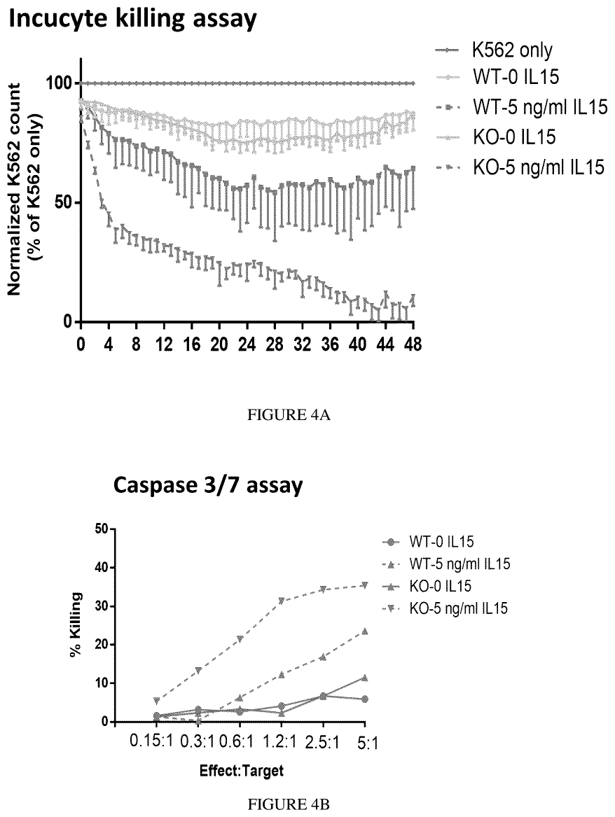

CISH is a potent intracellular inhibitory checkpoint in NK cell-mediated tumor immunity. Deletion of the CISH gene in human iPSC derived NK cells rendered the NK cells hypersensitive to cytokines thereby enhancing their cytotoxicity toward tumors (FIGS. 3A-4B). Compared with unmodified human NK cells, CISH knockout human NK cells will have better persistence and anti-tumor, anti-viral, and anti-microbial effects in human patient when used as cell source for adaptive cell therapy for treatment of cancers, viral and microbial infections.

The created CISH^{-/-} human iPSC-NK cells displayed a hypersensitive to IL-2/IL-15 stimulation and an ability to maintain expansion and anti-tumor functions with low concentration of IL-2 (5 U/ml) and IL-15 (5 ng/ml) (FIGS. 3A-3B). The CISH^{-/-} iPSC-NK cells could maintain expansion and cytotoxic function with low concentration of IL-2 (5 U/ml) and IL-15 (5 ng/ml) for more than 3 weeks in vitro.

Compared to existing art, the gene modified iPSC-derived NK cells have better anti-tumor effects as they can expand and persist longer than unmodified NK cells in vivo. Existing NK cell therapy uses unmodified NK cells, which are NK cells obtained from peripheral blood (PB-NK cells) or unmodified iPSC-derived NK cells, which typically require administration of high doses of IL-2 and/or IL-15 to maintain expansion and anti-tumor function. However, clinical data has been reported that high concentration of IL-2 and/or IL-15 has a high toxicity. Consequently, the CISH^{-/-} iPSC-derived NK cells can beneficially be used in NK cell therapy due to their mitigation of the toxicity caused by IL-2 and/or IL-15 by only requiring low doses of Il-2 and/or IL-15 or other cytokines to maintain expansion and anti-tumor function.

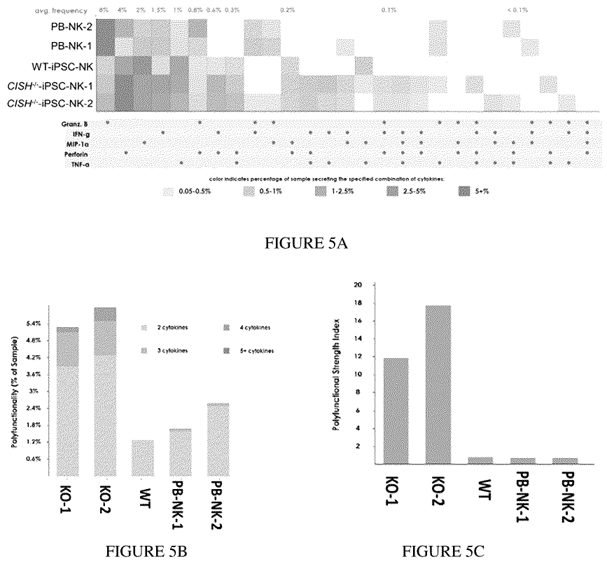

The CISH^{-/-} iPSC-derived NK cells show improved single-cell polyfunctionality. FIG. 5A shows a single-cell cytokine production analysis using the Isoplexis 32-plex, immune cytokine response panel, 5 effector cytokines (Granzyme B, IFNγ, MIP-1α, Perforin, TNFα) that are involved in cytotoxic functions. FIG. 5B shows a percentage of sample that secret two or more cytokines shown in FIG. 5A. FIG. 5C shows that polyfunctionality was measured through a polyfunctionality strength index (PSI), spanning a pre-specified panel of 32 key immunologically relevant molecules across major categories: homeostatic/proliferative, inflammatory, chemotactic, regulatory, and immune effector. Polyfunctionality of CAR-T cells (measured by Isoplexis 32-plex, same assay we used) were positively correlated with clinical outcome. The increase of polyfunctionality of CISH^{-/-} iPSC-NK cells explains better anti-tumor activities compared with unmodified wild-type NK cells.

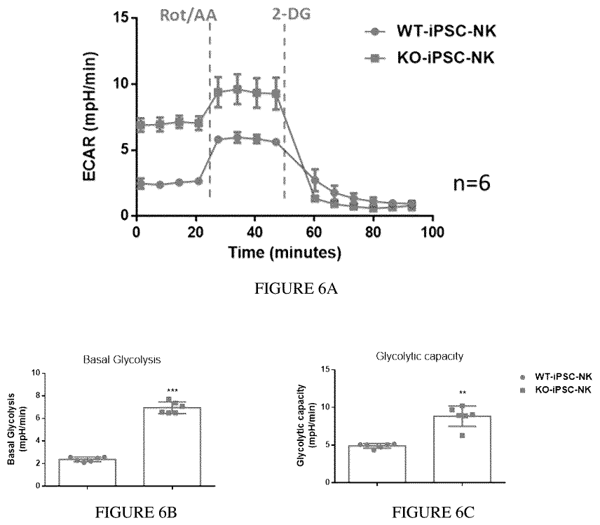

The CISH^{-/-} iPSC-NK cells show increased basal glycolysis and glycolytic capacity. FIG. 6A shows an extracellular acidification rate, (ECAR) was measured using Seahorse XF Glycolytic Rate Assay Kit. FIG. 6B shows quantification of basal glycolysis rate. FIG. 6C shows quantification of glycolytic capacity. Extracellular acidification rate (ECAR) is an indicator of glucose metabolism rate. This data shows that CISH^{-/-} iPSC-NK cells have improved glucose metabolism which may be the mechanism of improved functions of CISH^{-/-} iPSC-NK cells (improved glucose metabolism was reported to contribute to increased functions).

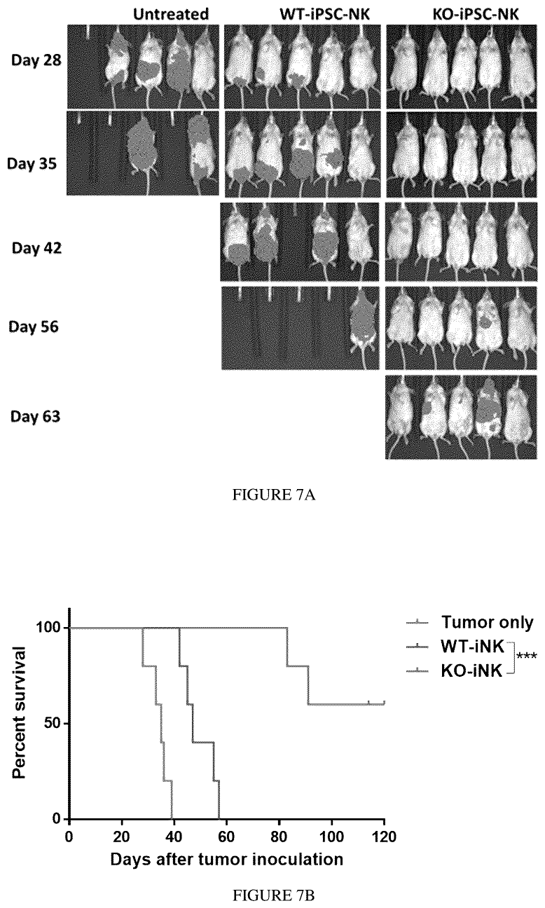

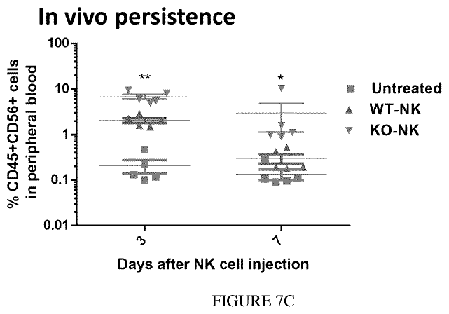

The CISH^{-/-} iPSC-NK demonstrate better anti-tumor activity in vivo NSG mice were inoculated IP with 5×10⁶ Molm13 cells expressing the firefly luciferase gene. 1 day after tumor transplant, mice were either left untreated or treated with 10×10⁶ WT-iPSC-NK or CISH KO-iPSC-NK cells. NK cells were supported by weekly injections of IL-2 for 3 weeks, and IVIS imaging was done weekly to track tumor load. FIG. 7A shows IVIS images. FIG. 7B shows the survival curve of each group. This data shows that CISHCISH^{-/-} iPSC-NK cells has improved anti-tumor activities in a xenograft tumor model.

In embodiments the CISH^{-/-} iPSC-derived NK cells, is used as an improved therapeutic cell source for NK cell therapies.

In embodiments the CISH^{-/-} iPSC-derived NK cells, are expanded in vitro to obtain a sufficient number of cells for administration as part of a treatment regimen of cancer, viral and microbial diseases, among other conditions.

In embodiments the CISH^{-/-} iPSC-derived NK cells are administered to a patient in a similar fashion to previous clinical work with NK cell-based therapies using unmodified peripheral blood NK cells. In embodiments, low concentrations of cytokine stimulation, such as with IL-2 and IL-15 are used as compared to conventional therapy with wtNK cells. In embodiments, the low concentration of IL-2 is between 1 and 10 U/ml, or about 5 U/ml, and the low concentration of IL-15 is between 1 and 10 ng/ml, or about 5 ng/ml, which is effective to maintain CISH^{-/-} NK cell expansion and anti-tumor functions.

In embodiments the CISH^{-/-} iPSC-derived NK cells are administered to a patient as part of a treatment regimen for refractory malignancies, such as but not limited to treating refractory cancers, both hematologic malignancies and solid tumors.

### Methods

### Hematopoiesis and NK Differentiation I:

CISH KO hiPSCs were differentiated first into hematopoietic progenitors and then into NK cells^{1, 2}. Briefly, upon the appearance of CD34⁺ cells inside the EB at day 6, EB was transferred into NK cell differentiation. Briefly, hematopoietic progenitors were transferred into NK cell differentiation medium containing a 2:1 mixture of Dulbecco modified Eagle medium/Ham F12 (Thermo Fisher Scientific, Waltham, MA, 11965092, 11765054), 2 mM L-glutamine (Thermo Fisher Scientific, Waltham, MA, 25030081), 1% penicillin/streptomycin (Thermo Fisher Scientific, Waltham, MA, 15140122), 25 μM β-mercaptoethanol (Thermo Fisher Scientific, Waltham, MA, 21985023), 20% heat-inactivated human serum AB (Corning, NY, U.S., MT35060CI), 5ng/mL sodium selenite (Merck Millipore, Burlington, MA, S5261), 50 μM ethanolamine (MP Biomedicals, ICN19384590), 20 mg/mL ascorbic acid (Merck Millipore, Burlington, MA, A4544), interleukin-3 (IL-3; R&D Systems Minneapolis, MN, 203-IL); for first week only), stem cell factor (SCF; R&D Systems Minneapolis, MN, 7466-SC), interleukin-15 (IL-15; R&D Systems, 247-ILB), Fms-like tyrosine kinase 3 ligand (FLT3L; R&D Systems Minneapolis, MN, 308-FK), and interleukin-7 (IL-7; R&D Systems Minneapolis, MN, 207-IL). The cells were then left in these conditions for 21 days receiving weekly media changes.

NK differentiation II:

After 21 days in NK differentiation medium (NK differentiation I), suspension cells were collected and transfer to 6-well plate with stromal cells OP9-DL4 (OP9 cells over-expressing DL4, Notch ligand) for 14 days receiving weekly media changes until they had developed into CD45+CD56+CD33-CD3- cells as determined by flow cytometry.

### Expansion

After differentiation, NK cells were expanded using the irradiated K562-IL21-4-1BBL^{3,4}. Briefly, non-adherent cells were removed and analyzed by flow cytometry to determine the purity of CD56+ NK cells. These cells were then stimulated with 2:1 aAPCs (irradiated at 10,000 Gy) to NK cells at 350,000 NK cells/mL of media containing RPMI 1640 (Thermo Fisher Scientific, Waltham, MA, 11875085), 2 mM L-glutamine (Thermo Fisher Scientific, Waltham, MA, 25030081), 1% penicillin/streptomyocin (Thermo Fisher Scientific, Waltham, MA, 15140122), 1% non-essential amino acids (NEAA; Thermo Fisher Scientific, Waltham, MA, 11140050) and 10% standard FBS or 10% human serum AB (Thermo Fisher Scientific, Waltham, MA, 10100147). This was supplemented with 50-100 U/mL IL2 (Prometheus, 65483011607).

### References

ADDIN EN.REFLIST 1. Knorr, D. A., Ni, Z., Hermanson, D., Hexum, M. K., Bendzick, L., Cooper, L. J., Lee, D. A. & Kaufman, D. S. (2013). Clinical-scale derivation of natural killer cells from human pluripotent stem cells for cancer therapy. Stem Cells Transl Med 2, 2013.
2. Zhu, H. & Kaufman, D. S. (2019). An improved method to produce clinical scale natural killer cells from human pluripotent stem cells. bioRxiv, 2019.
3. Denman, C. J., Senyukov, V. V., Somanchi, S. S., Phatarpekar, P. V., Kopp, L. M., Johnson, J. L., Singh, H., Hurton, L., Maiti, S. N., Huls, M. H., Champlin, R. E., Cooper, L. J. & Lee, D. A. (2012). Membrane-bound IL-21 promotes sustained ex vivo proliferation of human natural killer cells. PLOS One 7, 2012.
4. Hermanson, D. L., Bendzick, L., Pribyl, L., McCullar, V., Vogel, R. I., Miller, J. S., Geller, M. A. & Kaufman, D. S. (2016). Induced Pluripotent Stem Cell-Derived Natural Killer Cells for Treatment of Ovarian Cancer. Stem Cells 34, 2016.

What is claimed is:

1. A method for treating a disease in a human subject in need thereof, comprising administering to a human subject an effective amount of a pharmaceutical composition comprising human CISH^{-/-} natural killer (NK) cells and a pharmaceutically acceptable carrier.

2. The method of claim 1, wherein the human CISH^{-/-} NK cells are derived from induced pluripotent stem cells, embryonic stem cells, or peripheral blood cells.

3. The method of claim 1, wherein the CISH^{-/-} NK cells are autologous to the subject.

4. The method of claim 1, wherein the method further comprises administering to the subject an effective amount of one or more cytokines.

5. The method of claim 4, wherein the one or more cytokines comprise IL-2, IL-15, or a combination thereof, and the effective amount is less than an effective amount required with native NK cell treatment.

6. The method of claim 1, wherein the disease is hematopoietic cancer or a solid tumor.

7. The method of claim 1, wherein the disease is an infectious disease caused by a virus or microorganism.

8. The method of claim 1, wherein the human CISH^{-/-} NK cells are hypersensitive to cytokine stimulation and demonstrate improved expansion, anti-tumor function, and anti-viral function as compared to native NK cells.

9. A pharmaceutical composition comprising human CISH^{-/-} NK cells and at least one pharmaceutically acceptable excipient.

10. The pharmaceutical composition of claim 9, wherein the human CISH^{-/-} NK cells are derived from induced pluripotent stem cells, embryonic stem cells, or peripheral blood cells.

11. The pharmaceutical composition of claim 9, wherein the human CISH^{-/-} NK cells are derived from induced pluripotent stem cells.

12. The pharmaceutical composition of claim 9, wherein the human CISH^{-/-} NK cells are hypersensitive to cytokine stimulation and demonstrate improved expansion, antitumor function, and anti-viral function as compared to native NK cells.

13. The pharmaceutical composition of claim 12, wherein the cytokine stimulation comprises stimulation with IL-2 and/or IL-15.

14. A method for producing human CISH^{-/-} NK cells comprising:
a) deleting the CISH gene from human induced pluripotent stem cells (iPSCs), human embryonic stem cells (ESCs), or human peripheral blood cells (PBCs) to generate human CISH^{-/-} iPSCs, ESCs or PBCs; and
b) differentiating the CISH^{-/-} iPSCs, ESCs or PBCs into human CISH^{-/-} NK cells in vitro.

15. The method of claim 14, wherein the deletion of the CISH gene is achieved by using a CRISPR system.

16. The method of claim 14, wherein the differentiating step comprises a first differentiating step comprising differentiating the human CISH^{-/-} iPSCs, ESCs or PBCs into a cell population comprising at least 80% CD34⁺ cells, and a second differentiating step comprising differentiating the cell population comprising at least 80% CD34⁺ cells into a cell population comprising at least 80% CD45⁺ and CD56⁺ cells.

17. The method of claim 16, wherein the second differentiating step comprises contacting the cell population comprising at least 80% CD34⁺ cell with a Notch ligand.

18. The method of claim 17, wherein the Notch ligand is provided by OP9-DL4 cells.

19. A cell culture comprising human CISH^{-/-} NK cells.

20. The cell culture of claim 19, wherein the CISH^{-/-} NK cells are hypersensitive to cytokine stimulation and demonstrate improved expansion, anti-tumor function, and anti-viral function as compared to native NK cells.

21. A purified cell composition, comprising differentiated CISH^{-/-} induced pluripotent stem cells (iPSCs), wherein at least 70% of the cells in the composition are CD45+/CD56+ double-positive.

22. The composition of claim 21, wherein at least 80% of the cells in the composition are CD45+/CD56+ double-positive.

23. The composition of claim 21, wherein between 70% and 90% of the cells in the composition are CD45+/CD56+ double-positive.

24. The composition of claim 21, wherein between 80% and 90% of the cells in the composition are CD45+/CD56+ double-positive.

25. The composition of claim 21, wherein the iPSCs are human iPSCs.

26. The composition of claim 21, provided that the iPSCs do not express a chimeric antigen receptor.

27. The composition of claim 21, provided that the iPSCs do not express an exogenous IL-15.

28. The composition of claim 21, provided that the iPSCs do not express an exogenous IL-2.

30. The method of claim 1, wherein the human CISH^{-/-} natural killer (NK) cells are derived from human induced pluripotent stem cells (iPSCs).
